(19) United States
(12) Patent Application Publication
(10) Pub. No.: US 2012/0252045 A1
(43) Pub. Date: Oct. 4, 2012

(54) METHOD OF ASSESSING ISLET FUNCTION

(76) Inventor: Andrew Pepper, Edmonton (CA)

(21) Appl. No.: 13/393,587

(22) PCT Filed: Sep. 3, 2010
(86) PCT No.: PCT/IB2010/002369

§ 371 Date: Jun. 19, 2012

Related U.S. Application Data
(60) Provisional application No. 61/239,679, filed on Sep. 3, 2009.

(51) Int. Cl. C12Q 1/02; C12M 1/34; G06F 19/24; C12Q 1/54

(52) U.S. Cl. 435/14; 435/29; 435/287.5; 702/19

(57) ABSTRACT

A method for assessing islet function comprising determining an oxygen consumption rate (OCR) of the islets; determining an islet index (II) of the islets; and determining an OCR/II ratio, wherein a high OCR/II ratio correlates with increased islet function. The methods may be performed on islets in vitro. In some embodiments, the methods may be performed on islets to be used for transplantation after they are harvested from a donor pancreas and prior to transplanting the islets into a recipient.

This application claims priority to U.S. Provisional Application No. 61/239,679, filed Sep. 3, 2009, which is incorporated herein by reference in its entirety.

This application relates generally to methods for assessment of the function of islet cells.

The therapeutic value of islet transplantation as a treatment for diabetes remains controversial despite significant improvements over the past decade. However, replacement or regeneration of insulin-secreting β-cells to restore carbohydrate control continues to be a focus of diabetes research. A major consideration for any transplant, including islet cell transplants, is the functional quality of the graft, which can be influenced by such variables as the condition of the donor pancreas, preservation technique, and ischaemic times. In addition, islet damage caused during isolation of the islets from the pancreas can also affect transplant function.

Currently there are limited in vitro pre-transplant techniques for predicting the post transplant function of isolated islets. Proposed methods of in vitro assessment of the quality of an islet preparation include measurement of various indicators of cell function including insulin secretion levels in response to glucose challenge, oxygen consumption rate, ATP to ADP ratios, action potentials and, more simplistically, viability, size, and/or number or packed cell volume. However, none of these methods has proved to be a reliable indicator of in vivo function, and there is currently no reliable in vitro, pre-transplant technique for predicting the post transplant function of isolated islets.

FDA guidelines recommend an in vivo diabetic immunodeficient (athymic or SCID) mouse bioassay to test the function of islets used for clinical transplantation. However, this assay is costly and technically demanding, and the results of this assay are retrospective. In addition, the minimum number of islets equivalents (IEQ) required to reverse diabetes in the athymic mouse is approximately 2,000 (100,000 IEQ/kg), which is vastly in excess of the 100 IEQ (5000 IEQ/kg) required to reverse diabetes using an isograft in mice. This discrepancy is probably due to the insensitivity of rodents to both porcine and human insulin, and consequently, the number of islets needed to achieve normal blood glucose levels in rodents does not correlate well with the number needed to treat humans.

Accordingly, there is a need for methods of assessing the function and/or viability of islets. Therefore, in various embodiments, a novel method to predict the viability of islets prior to transplant, the ability of islets to improve blood glucose levels post transplant, and/or the ability of islets to reverse diabetes is provided.

In certain embodiments, a method for assessing islet function in vitro is provided. The method can comprise determining an oxygen consumption rate (OCR) of a group of islet cells; determining an islet index (II) of the islet cells; and determining an OCR/II ratio. In some embodiments, the OCR may be measured in nmol/min/mg-DNA, and the islet index may be determined by dividing the islet equivalent number (IEQ) by the actual number of islets, indicating the size distribution of the preparation.

In some embodiments, the method further comprises determining whether or not the islets are suitable for implantation in a host based on the OCR/II ratio. In some embodiments, islets having an OCR/II ratio within a range of about 50 nmol per min per mg DNA/islet index to about 250 nmol per min per mg DNA/islet index are identified as being suitable for transplantation. In other embodiments, islets having an OCR/II ratio of about 50 nmol per min per mg DNA/islet index or more, or about 60 nmol per min per mg DNA/islet index or more, or about 70 nmol per min per mg DNA/islet index or more, or about 80 nmol per min per mg DNA/islet index or more, or about 90 nmol per min per mg DNA/islet index or more, or about 100 nmol per min per mg DNA or more, or about 120 nmol per min per mg DNA/islet index or more, or about 140 nmol per min per mg DNA/islet index or more, or about 160 nmol per min per mg DNA/islet index or more, or about 180 nmol per min per mg DNA or more, or about 200 nmol per mg DNA or more, or about 250 nmol per mg DNA,or any ranges between those values are identified as being suitable for transplantation. In some embodiments these OCR/II ranges may be adjusted depending on maturity of the cells (e.g. islets from neonatal, immature or adults animals), degree of differentiation, and species. In some embodiments, the islets are adult islets. In other embodiments, the islets are immature islets.

In some embodiments, a method of assessing islet cell function is provided. In some embodiments, the method comprises selecting a group of islets for potential transplantation into a host; selecting a portion of the islets to assess the suitability of the group of islets for transplantation; determining an oxygen consumption rate (OCR) of the portion of the group of islet cells; determining an islet index(II) of at least the portion of the group of islet cells; and determining whether or not the islets are suitable for implantation in a host based on the OCR/II ratio. In some embodiments, islets having an OCR/II ratio within a range of about 50 nmol per min per mg DNA/islet index to about 250 nmol per min per mg DNA/islet index are identified as being suitable for transplantation. In other embodiments, islets having an OCR/II ratio of about 50 nmol per min per mg DNA/islet index or more, or about 60 nmol per min per mg DNA/islet index or more, or about 70 nmol per min per mg DNA/islet index or more, or about 80 nmol per min per mg DNA/islet index or more, or about 90 nmol per min per mg DNA/islet index or more, or about 100 nmol per min per mg DNA/islet index or more, or about 120 nmol per min per mg DNA/islet index or more, or about 140 nmol per min per mg DNA/islet index or more, or about 160 nmol per min per mg DNA/islet index or more, or about 180 nmol per min per mg DNA/islet index or more, or about 200 nmol per min per mg DNA/islet index or more, or about 250 nmol per min per mg DNA/islet index, or any ranges between those values are identified as being suitable for transplantation. In some embodiments these OCR/II ranges may be adjusted depending on cell maturity, differentiation, and species. In some embodiments, the islets are adult islets. In other embodiments, the islets are immature islets.

In various embodiments, a high OCR/II ratio correlates with improved islet function in vivo regardless of cellular species of origin, cell maturity, or differentiation. In some embodiments, islet function is measured as viability of the islets. In other embodiments, islet function is measured as ability of the islets to reverse diabetes or to improve blood glucose levels in a recipient of the islets. In some embodiments, the methods are performed in vitro. In some embodiments, the methods may be performed on islets to be used for transplantation after they are harvested from a donor pancreas and prior to transplanting the islets into a recipient.

In various embodiments, the present disclosure also provides devices for automated assessment of islet function. For example, in certain embodiments a device for assessing islet cell function is provided. In various embodiments, the device comprises a first cell analysis unit configured to determine an oxygen consumption rate (OCR) of a group of islet cells; a second cell analysis unit configured to determine the cellular size distribution, as measured as an islet index (II) of the islet cells; and a computation unit configured to determine an OCR/II ratio, wherein a high OCR/If ratio correlates with improved islet function in vivo.

### BRIEF DESCRIPTION OF THE DRAWINGS

FIG. 1 depicts the correlation of a viability stain assay performed in vitro with rates of diabetes reversal in nude Balb/c mice post porcine islet xenograft, as described in Example 1.

FIG. 2 depicts the correlation of a glucose stimulation index measured in vitro with rates of diabetes reversal in nude Balb/c mice post porcine islet xenograft, as described in Example 2.

FIG. 3 depicts the correlation of an islet index, which relates to islet size, measured in vitro with rates of diabetes reversal in nude Balb/c mice post porcine islet xenograft, as described in Example 3.

FIG. 4 depicts the correlation of an oxygen consumption rate measured in vitro with rates of diabetes reversal in nude Balb/c mice post porcine islet xenograft, as described in Example 4.

FIG. 5A is a bar graph demonstrating OCR inhibition of islet cells using sodium azide (NaAz). The NaAz inhibition was performed to validate OCR measurement data described in Example 4.

FIG. 5B is a bar graph demonstrating OCR inhibition of βTC6 cells using sodium azide (NaAz). The NaAz inhibition was performed to validate OCR measurement data described in Example 4.

FIG. 6 depicts the correlation of the ratio of oxygen consumption rate (OCR) per mg of DNA/islet Index (OCR/II) with rates of diabetes reversal in nude Balb/c mice post porcine islet xenograft, as described in Example 6.

FIG. 7A illustrates a logistic regression analysis showing the probability of islet graft survival based on the OCR/II ratio, as described in Example 6.

FIG. 7B provides ROC analysis of the data of FIG. 7A.

FIG. 8 graphically illustrates blood glucose levels vs. days post transplant for animals having an OCR/II>70 nmol/mg-DNA or <70 nmol/mg-DNA, as described in Example 6.

FIG. 9 illustrates the relationship between the OCR/II ratio measure in vitro for functioning and non-functioning porcine-to-porcine grafts, as described in Example 7.

FIG. 10 illustrates a system for assessing islet function, according to certain embodiments.

### EXEMPLARY EMBODIMENTS

Reference will now be made in detail to embodiments of this disclosure, examples of which are illustrated in the accompanying drawings.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

In various embodiments, a method for assessing the function of islets is provided. The method can be performed in vitro before implantation in a recipient. The method can comprise determining an oxygen consumption rate (OCR) of a group of islet cells; determining an islet index (II) of the islet cells; and determining an OCR/II ratio. In some embodiments, the OCR may be measured in nmol per min per mg DNA, and the islet index may be determined by dividing the islet equivalent number (IEQ) by the actual number of islets, indicating the size distribution of the preparation.

In some embodiments, the method further comprises determining whether or not the islets are suitable for implantation in a host based on the OCR/II ratio. In some embodiments, islets having an OCR/II ratio within a range of about 50 nmol per min per mg DNA/islet index to about 250 nmol per min per mg DNA/islet index are identified as being suitable for transplantation. In other embodiments, islets having an OCR/II ratio of about 50 nmol per mg min per DNA/islet index or more, or about 60 nmol per min per mg DNA/islet index or more, or about 70 nmol per min per mg DNA/islet index or more, or about 80 nmol per min per mg DNA/islet index or more, or about 90 nmol per min per mg DNA/islet index or more, or about 100 nmol per min per mg DNA/islet index or more, or about 120 nmol per min per mg DNA/islet index or more, or about 140 nmol per min per mg DNA/islet index or more, or about 160 nmol per min per mg DNA/islet index or more, or about 180 nmol per min per mg DNA/islet index or more, or about 200 nmol per min per mg DNA/islet index or more, or about 250 nmol per min per mg DNA/islet index are identified as being suitable for transplantation. In some embodiments, the islets are adult islets. In other embodiments, the islets are immature islets.

In some embodiments, a method of assessing islet cell function is provided. In some embodiments, the method comprises selecting a group of islets for potential transplantation into a host; selecting a portion of the islets to assess the suitability of the group of islets for transplantation; determining an oxygen consumption rate (OCR) of the portion of the group of islet cells; determining an islet index (II) of at least the portion of the group of islet cells; and determining whether or not the islets are suitable for implantation in a host based on the OCR/II ratio. In some embodiments, an OCR/II ratio within a range of about 50 nmol per min per mg DNA/islet index to about 250 nmol per min per mg DNA/islet index are identified as being suitable for transplantation. In other embodiments, an OCR/II ratio of about 50 nmol per min per mg DNA/islet index or more, or about 60 nmol per min per mg DNA/islet index or more, or about 70 nmol per min per mg DNA/islet index or more, or about 80 nmol per min per mg DNA/islet index or more, or about 90 nmol per min per mg DNA/islet index or more, or about 100 nmol per min per mg DNA/islet index or more, or about 120 nmol per min per mg DNA/islet index or more, or about 140 nmol per min per mg DNA/islet index or more, or about 160 nmol per min per mg DNA/islet index or more, or about 180 nmol per min per mg DNA/islet index or more, or about 200 nmol per min per mg DNA/islet index or more, or about 250 nmol per min per mg DNA/islet index are identified as being suitable for transplantation. In some embodiments, the islets are adult islets. In other embodiments, the islets are immature islets.

The OCR for a group of islets can be determined in a number of ways. In certain embodiments, the OCR is measured by measuring the decrease in pO₂ in a sealed area having a known oxygen concentration and/or known oxygen flow. In certain embodiments, the method includes placing the islets in an isolated atmosphere and measuring the change in pO₂ in the atmosphere. Such measurements can be performed using a fiber optic sensor oxygen monitoring system (Instech Laboratories Plymouth Meeting, Pa.). For example, one suitable method for measuring OCR is described by Papas et al., “A Stirred Microchamber for Oxygen Consumption Rate Measurements with Pancreatic Islet Cells,” Biotechnol Bioeng 2007; 98: 1071-1082. Any suitable method for measure OCR for a group of islets can be used.

The islet index (which is also sometimes called the isolation index), can be calculated in a number of ways. Generally, methods employed to calculate islet index initially involve quantifying islet yield, and the actual number of islets counted are converted to islet equivalents (IEQ) by standardizing islets to an average of 150 μm in diameter. See, Ricordi, C. et al., “Islet Isolation Assessment in Man and Large Animals,” Acto Diabetol Lat 1990; 27: 185-195. The islet index (or isolation index) is calculated as the ratio of IEQs to the actual number of islets quantified.

In various embodiments, one skilled in the art could use islet enzymatic dissociation to select smaller cells/groups of cells or other methodologies to select small clusters of cells. See Ichii et al., “A Novel Method for the Assessment of Cellular Composition and Beta-Cell Viability in Human Islet Preparations,” Am J Transplant 57(7): 1635-1645 (2005).

Islet of Langerhans can be diverse in size with respect to diameter ranging from 50 microns to >400 microns. Therefore, an Islet Equivalent (IEQ) represents a standardized measure of an islet based on a typical size equal to 150 micron. Islets are classified into eight classes based on their diameter, and each class contains its own multiplication factor standardized to 150 microns—thus an islet that has a diameter or 150 microns is equal to 1 IEQ, whereas an islet that has a diameter of 400 microns (class 8) would be equal (or “equivalent”) to 20 IEQ.

As noted above, in certain embodiments, the OCR can be standardized to the number of cells present. In certain embodiments, the OCR is determined based on the amount of DNA and is expressed as nmol/min/mg-DNA or equivalent units. The OCR may also be expressed as an OCR per number of cells or other unit representative of the number of cells. Devices and methods for quantifying DNA are known in the art and include, for example, Quant-iT PicoGreen dsDNA Dit (Molecular Probes, Eugene, Oreg.).

In various embodiments, a high OCR/II ratio correlates with improved islet function in vivo. In some embodiments, islet function is measured as viability of the islets. In other embodiments, islet function is measured as ability of the islets to reverse diabetes or to improve blood glucose levels in a recipient of the islets. In various embodiments, reversal of diabetes may be defined as non-fasting blood glucose below 11.1 mmol/L, or fasting blood glucose 7.8 mmol/L, or equivalent units. In various embodiments, improved glucose control can be defined as a reduction in Hemoglobain A1C (HbA1C) compared to pre-transplant values, an increase in serum C-peptide concentrations post transplant compared to pre-transplant values, improved response to a glucose tolerance test as measured by area under curve for glucose, insulin and c-peptide, glucose disappearance rates, and any of the American Diabetes Association criteria for diabetes (guidelines determined by the American Diabetes Association “Report of Expert Committee on the Diagnosis and Classification of Diabetes Mellitus. Diabetes Care 1997; 1183-97).

In some embodiments, the methods are performed in vitro. In some embodiments, the methods may be performed on islets to be used for transplantation after they are harvested from a donor pancreas and prior to transplanting the islets into a recipient. The methods are useful, for example, as in vitro assays of islets to determine the ability of a sample of islets to function either in further in vitro studies or in vivo, for example, in islet transplants for the treatment of diabetes. The methods of this application are also useful in determining a threshold parameter that is predictive of islet function and for preselecting of viable tissues to be transplanted.

In one aspect, the methods provide an efficient, inexpensive, and predictive method of determining islet function. For example, methods of determining an OCR/II ratio require a minimum amount of islets, for example about 1,000 to about 3,000 IEQ, fewer than 3,000 IEQ, fewer than 2,000 IEQ, or fewer than 3,000 IEQ; and can be determined in a short period of time. certain embodiments, one skilled in the art could also use about 100 IEQ to 1000 IEQ, about 100 IEQ to 300 IEQ, or about 300 IEQ to about 1000 IEQ.

In some cases, the OCR/II ratio can be determined within about 30 minutes. Thus, the methods of the invention may be used to predict islet function without consuming a large amount of islets. This is useful in the case of islet transplants, which require conservation of high quality, clinical grade islets for the transplant procedure and where time and efficiency of culture is important in enhancing islet survival. In some embodiments, an OCR/II ratio of about 70 or more indicates that the islets have an increased ability to function. In some embodiments an OCR/II ratio between 50 and 70 nmol per min per mg DNA/islet index indicates a medium ability of islets to function, while OCR/II ratios below 50 indicate a low ability of islet to function. In various embodiments, for example, where different species, maturity, and differentiation of cells are used, these ranges may be adjusted.

The islets to be assessed using the above-discussed methods can include a variety of different islet sources. For example, in various embodiments, the islets can include islets isolated from non-human sources, including pigs, mice, rats, or other animals. Furthermore, insulin-producing cells can be derived from non-primary cell lines such as stem cells. In addition, the islets can be autogenic, allogenic, or xenogenic to the intended recipient. In certain embodiments, the recipient is a human and the islets are autogenic, allogenic, or xenogenic. In other embodiments, the recipient is an animal, which may be treated for diabetes and/or may be used in research. In still other embodiments, islets assessed using the methods discussed above are not to be implanted, but are merely assessed to evaluate various islet procurement, treatment, preservation, culture, or isolation procedures. In various embodiments, the islets comprise at least one of human islets, non-human animal islets, stem cells, genetically modified cells, or any cell which releases insulin.

Further, although various threshold OCR/II values are provided, such values may be affected by other factors, and the methods of the present disclosure can readily be used to assess any group of islets. For example, the islet OCR/II ratio can be affected by storage solutions, isolation protocols, environmental conditions, islet source, etc. For example, islets from one source may have different sizes than islets from another source. The methods of the present disclosure can readily be adapted for assessment of islet functionality using islets affected by such variables, and OCR/II values applicable to such islets can be ascertained.

In various embodiments the islets tested may be then placed in the body in a subcutaneous or intraperitoneal location. Without limitation, the islets may be placed in or associated with any tissue or organ in the body, e.g. kidney capsule, omentum, skin, digestive organs, e.g. intestines, stomach, bowel, or secretory organs, e.g. pancreas, gall bladder. The islets may be placed in a prevascularized chamber, or within a polymer or non-polymer formulation or infused into the portal vein of the liver or other vessel or a combination thereof. In one embodiment, the islets may be encapsulated in a polymer before being placed in the body.

In some embodiments, the OCR/II ranges discussed above may be adjusted depending on cell maturity, differentiation, and species. For example, in some embodiments, OCR/II values greater than 10 nmol/min-mgDNA, or greater than 20 nmol/min-mgDNA, or greater than 30 nmol/min-mgDNA are suitable for implantation. For example, as shown below in Example 8, immature islets having an OCR/II ratio less than 50 nmol/min-mgDNA can be suitable for producing functioning grafts in some animals.

Devices, Cells, and Kits:

In various embodiments, the present disclosure also provides devices for automated assessment of islet function. For example, in certain embodiments, a device for assessing islet cell function is provided. In various embodiments, the device comprises a first cell analysis unit configured to determine an oxygen consumption rate (OCR) of a group of islet cells; a second cell analysis unit configured to determine an islet index (II) or size distribution of the islet cells; and a computation unit configured to determine an OCR/II ratio, wherein a high OCR/II ratio correlates with improved islet function in vivo. Suitable devices can be incorporated in automated cell production systems, and may be contained in a common housing or as separate components.

FIG. 10 illustrates a system 10 for assessing islet function, according to certain embodiments. As shown, the system 10 includes a housing or chamber in which a group of islets 20 may be placed for analysis. The system 10 further includes first analysis unit 30, which can comprise an oxygen sensor for measuring an oxygen consumption rate of the islets 20. In addition, the system 10 can include a second analysis unit 40 configured to measure an islet index of the islets.

As shown, the system 10, comprising the first 30 and second 40 analysis units can be a single integrated system with a common housing. However, the first and second analysis units may be contained in separate housings, and the islets may be transferred to the separate housings to perform OCR calculations and/or islet index measurements. The transfer may be performed manually or by an automated system. In addition, in certain embodiments, the one or both of the OCR and islet index measurements may be performed by an operator, such as a technician. For example, the system 10 may measure the OCR automatically having placement of islets 20 in the system 10, and an operator may calculate the islet index may manual inspection.

In some embodiments, islets or other insulin-producing cells produced and/or tested according to the methods of the present disclosure are provided. As noted above, the cells can be derived from a variety of different sources. For example, in various embodiments, the cells can include islets isolated from non-human sources, including pigs, mice, rats, or other animals. Furthermore, insulin-producing cells can be derived from non-primary cell lines such as stem cells. In addition, the cells can be autogenic, allogenic, or xenogenic to the intended recipient. In certain embodiments, the recipient is a human and the cells are autogenic, allogenic, or xenogenic.

After isolation, culture, and/or before and/or after storage (e.g., by cryopreservation), the cells may be assessed to determine their suitability for implantation in a recipient. In some embodiments, the cells are assessed by selecting a portion of the cells to assess the suitability of the group of cells for transplantation; determining an oxygen consumption rate (OCR) of the portion of the group of cells; determining an islet index(II) of at least the portion of the group of cells; and determining whether or not the cells are suitable for implantation in a host based on the OCR/II ratio. In some embodiments, adult islets having an OCR/II ratio within a range of about 50 nmol per min per mg DNA/islet index to about 250 nmol per min per mg DNA/islet index are identified as being suitable for transplantation. In other embodiments, islets having an OCR/II ratio of about 50 nmol per min per mg DNA/islet index or more, or about 60 nmol per min per mg DNA/islet index or more, or about 70 nmol per min per mg DNA/islet index or more, or about 80 nmol per min per mg DNA/islet index or more, or about 90 nmol per min per mg DNA/islet index or more, or about 100 nmol per min per mg DNA/islet index or more, or about 120 nmol per min per mg DNA/islet index or more, or about 140 nmol per min per mg DNA/islet index or more, or about 160 nmol per min per mg DNA/islet index or more, or about 180 nmol per min per mg DNA/islet index or more, or about 200 nmol per min per mg DNA/islet index or more, or about 250 nmol per min per mg DNA/islet index, or any ranges between those values are identified as being suitable for transplantation.

In certain embodiments, cryopreserved cells are provided. In certain embodiments, the cells include groups of islets cells that have been isolated. A portion of the isolated islets cells are assessed to determine their suitability for implantation based on their OCR/II, and cells that are suitable for implantation are cryopreserved.

In certain embodiments, kits comprising islets or other insulin-producing cells are provided. The kits can include isolated islets or other cells that have been selected for potential implantation in a host. The kits can further include instructions for assessing the cells to determine their suitability for implantation. In some embodiments, the cells are to be assessed by selecting a portion of the cells to assess the suitability of the group of cells for transplantation; determining an oxygen consumption rate (OCR) of the portion of the group of cells; determining an islet index(II) of at least the portion of the group of cells; and determining whether or not the cells are suitable for implantation in a host based on the OCR/II ratio. In various embodiments, the kit can include instructions for using the cells to treat insulin-dependent diabetes. In some embodiments, the instructions will indicate that cells having an OCR/II ratio within a range of about 50 nmol per min per mg DNA/islet index to about 250 nmol per min per mg DNA/islet index are suitable for transplantation. In other embodiments, the instructions will indicate that cells having an OCR/II ratio of about 50 nmol per min per mg DNA/islet index or more, or about 60 nmol per min per mg DNA/islet index or more, or about 70 nmol per min per mg DNA/islet index or more, or about 80 nmol per min per mg DNA/islet index or more, or about 90 nmol per min per mg DNA/islet index or more, or about 100 nmol per min per mg DNA/islet index or more, or about 120 nmol per min per mg DNA/islet index or more, or about 140 nmol per min per mg DNA/islet index or more, or about 160 nmol per min per mg DNA/islet index or more, or about 180 nmol per min per mg DNA/islet index or more, or about 200 nmol per min per mg DNA/islet index or more, or about 250 nmol per min per mg DNA/islet index, or any ranges between those values are suitable for transplantation.

It is to be understood that both the foregoing general description and the following examples are explanatory only and are not restrictive of the invention, as claimed.

### EXAMPLES

### Examples 1-7

### Determination of the Correlation of Various In Vitro Measurements with the Ability of Porcine Islets to Reverse Diabetes in Athymic Mice

In this study we examined five different in vitro parameters to assess the ability of those parameters to predict in vivo function. The parameters included viability staining, a glucose stimulation index, an islet index, an oxygen consumption rate, and an oxygen consumption rate standardized to an islet index. The various indices are described in more detail below.

The examples were generated from adult porcine islet isolations, each of which was tested for in vivo function by transplantation under the kidney capsule of 3-6 diabetic athymic mice. Mice that were considered sick either at the time of transplant or immediately post transplant were euthanized and have been excluded from analysis, as were any mice, which at the time of transplant, had a blood glucose level of less than 18 mM. At the end of the observation period (100 days) all normoglycaemic mice were nephrectomized to confirm the restoration of hyperglycaemia, which occurred in every case.

The assays and transplants performed in Examples 1-7 consumed approximately 10% of the islets from each isolation. Given the scarcity of human pancreata, it was considered inappropriate to perform these studies using clinical grade islet isolations and unrepresentative to use only islets from isolations that failed to meet clinical release criteria. However, porcine islets provide a stringent surrogate for the clinical environment. Once porcine islets are isolated, their function is comparable to that of human islets.

Porcine Pancreatic Islet Isolation

Adult porcine islets were isolated from female (>2 years) Yorkshire-Landrace pigs using a modified Ricordi technique yielding >90% purity from exocrine tissue. Ricordi, C. et al., “Islet Isolation Assessment in Man and Large Animals,” Acto Diabetol Lat 1990; 27: 185-195. Porcine islets were cultured overnight in modified RMPI (10% FBS, 5 mM Nicotinamide, 2 mM Glutamax, 1% P/S) at 37° C. Post culture, islets were counted, assayed for functional capacity, and transplanted into diabetic athymic mice.

Correlation of In Vitro Assays to In Vivo Islet Function:

To correlate in vitro measurements with the ability of islets to restore normoglycaemia, islets were transplanted into diabetic nude mice. Diabetes was induced in 15-20 g male athymic Balb/c nude mice (Charles River, Wilmington, Mass.) by IP injection of 200 mg/kg streptozotocin (STZ) (Sigma Chemicals), freshly reconstituted in citric acid/citrate buffer (pH 4.5-4.7). Diabetes was defined as two non-fasting blood glucose readings of >18 mmol/L at least two days apart.

Following, porcine islet isolation and culture (described above), 4,000 IEQ were transplanted under the left renal capsule of the diabetic nude mice. Blood glucose was monitored using a mini glucometer (Freestyle) on days 0 (time of transplant), 1, 4 and 7, followed by weekly blood glucose recordings. Animals in which diabetes was reversed were nephrectomized, and blood glucose levels were monitored to confirm restoration of hyperglycaemia.

Statistical Analysis

The differences between groups were assessed by paired t-test and by ANOVA. The individual predictive ability of each pre-transplant assay was assessed using a logistic regression model with probability of diabetic reversal as response. P<0.05 was considered to be statistically significant. Since each data point is generated from a number of different islet isolations, the results are reported as mean of each individual experiment±SD.

The individual predictive abilities of viability staining, glucose stimulation index, islet size, oxygen consumption rate, and standardized oxygen consumption were assessed using a logistic regression model with probability of diabetic reversal as response. A P-value of 0.05 was used to declare the statistical significance of an index in predicting probability of reversal. The extent to which a predictor distinguishes between reversal and non-reversal was investigated using the receiver operation characteristic curve (ROC) methodology. Data analysis was performed using SAS 9.1 (SAS institute, Cary, N.C.).

### Example 1

### Viability Staining of Islets

Viability stains are based on dye exclusion, which demonstrates membrane integrity. Cells that have intact plasma membranes will tend to exclude DNA chelating dyes from entering the cells, while cells that have damaged plasma membranes will stain with the DNA chelating dye. However, viability stains do not assess metabolic function.

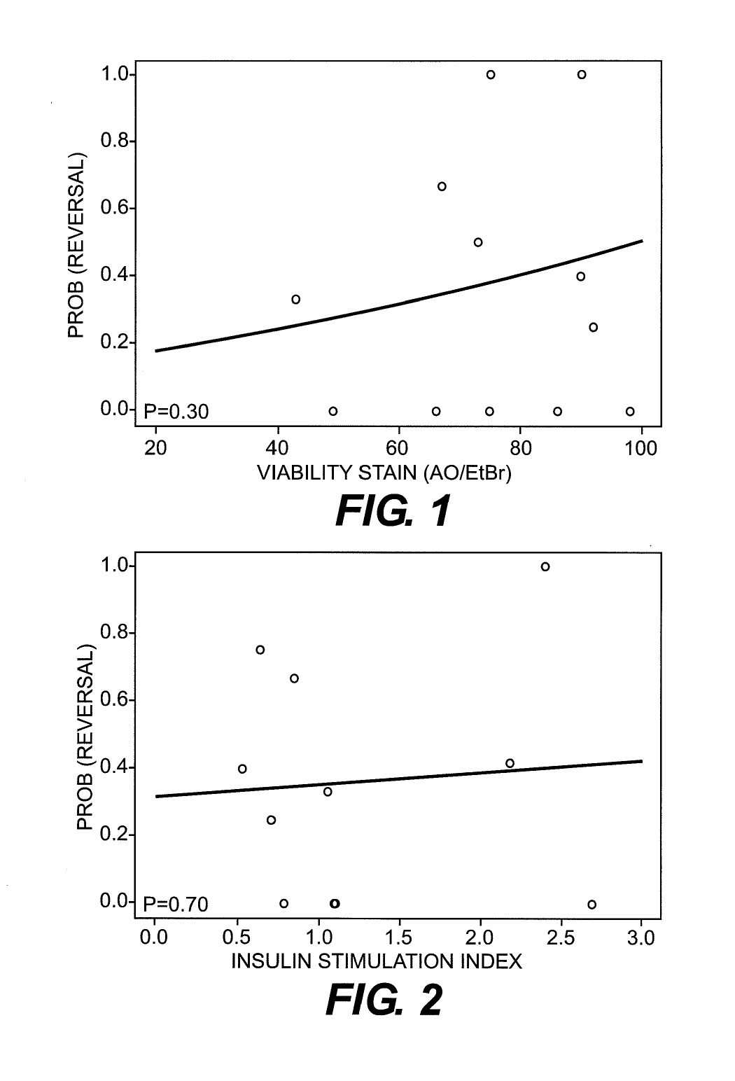

Briefly, three aliquots of 100 IEQ were stained with acridine orange/ethidium bromide to assess viability. Each aliquot of the islet suspension was assayed using a fluorescence microscope with a combined filter of 647 nm/535 nm. Twelve islet isolations were tested in a total of 45 transplants for the ability of viability staining to predict in vivo reversal of diabetes. ROC analysis showed that this assay is not significantly better than chance at predicting reversal with an area under the curve of 0.59 with 95% confidence limits of 0.42-0.76. Groups of islets that stained with greater than 90% viable produced in a rate of diabetes reversal of only 43.1%. While there is a strong correlation (R2=0.9200) between viability and increase in diabetes reversal, logistic regression analysis shows (FIG. 1) that this parameter is not useful as a predictor of islet function; P=0.30, meaning there is no evidence to suggest staining viability be a useful predictor for probability of reversal of diabetes.

### Example 2

### Glucose Stimulation Index

The ability of isolated islets to secrete insulin in response to glucose was measured. Briefly, aliquots of 100 IEQ (three replicates) were incubated in 1.5 ml of RPMI containing 2.8 mmol/L glucose (low) for 1 hour at 37° C. Simultaneously, 100 IEQ (three replicates) were incubated with 1.5 ml fresh RPMI containing 20 mmol/L glucose (high) for 1 hour at 37° C. Supernatants from all cultures were removed, and porcine insulin levels in the supernatants were measured using ELISA (Mercodia AB) according to the manufacturer's instructions. The DNA content of the islet pellets was measured (Qiagen DNeasy DNA isolation kit), and the quantity of insulin secreted (μg/L) was standardized to the amount of DNA present (mg). The ratio of the mean concentrations of insulin secreted at high to low glucose levels was calculated, and that ratio was considered the stimulation index (SI).

Islets from twelve different isolations were tested in vitro for their ability to produce insulin in response to a glucose challenge. The results of this assay did not correlate with subsequent reversal of diabetes in 53 transplants with an AUC from ROC analysis of 0.54 with confidence limits of 0.37-0.71 and no significant (P=0.7) logistic regression correlation (FIG. 2). These results indicate that the ability of islets to respond to glucose was of no value in predicting post transplant outcome.

### Example 3

### Islet Index

The islet index (II) provides an indication of the size distribution of islets in an isolation. The islet index is derived by dividing the total number of IEQ by the actual number of islets (range 50-400 μm). Thus, a large islet index indicates a greater proportion of islets over 150 μm present in the islet isolation.

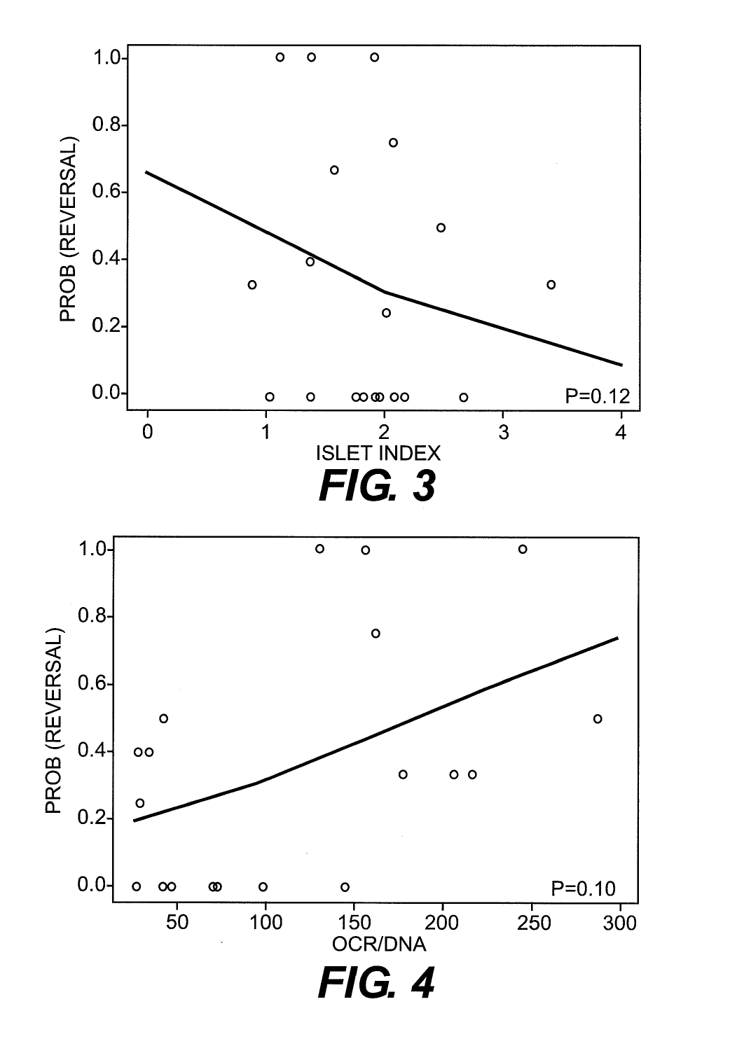

Logistical regression analysis of seventy-seven transplants was performed to determine if islet size affects diabetes reversal rates. The analysis indicated that nude mice that were transplanted with the greatest number of small islets, became normoglycaemic with the highest frequency (FIG. 3). However, ROC analysis indicated that as a diagnostic test of function, islet size is not significantly better than chance with an AUC of 0.68 and 95% confidence limits of 0.49 to 0.76. These data suggest that smaller islets are more efficient at reversing diabetes. However, islet size is of marginal value (P=0.12) in predicting islet function (FIG. 3).

### Example 4

### Measurement of Oxygen Consumption Rates (OCR)

Porcine islet OCRs were assessed in triplicate aliquots of 1000 IEQ. Controls comprising media alone and heat-killed islets (1000 IEQ ×3 incubated for 1 hour at 60° C.) were assayed in parallel. OCRs were measured using a fiber optic sensor oxygen monitoring system (Instech Laboratories Plymouth Meeting, Pa.), which quantifies the decrease in oxygen partial pressure (pO₂) over time, as described by Papas et al., “A Stirred Microchamber for Oxygen Consumption Rate Measurements with Pancreatic Islet Cells,” Biotechnol Bioeng 2007; 98: 1071-1082. OCRs were standardized to the amount of DNA in each chamber (nmol/min-mgDNA), which was determined using a Quant-iT PicoGreen dsDNA kit (Molecular Probes, Eugene, Oreg.). The validation of this assay is described in Example 5.

While logistic regression analysis (FIG. 4) of 20 islet isolations failed to achieve statistical significance (P=0.10), the results show a superior trend compared to the other assays tested (FIGS. 1-3). ROC analysis of seventy-seven transplants indicated that this assay is significantly better (P=0.004) than random chance at predicting reversal with an AUC of 0.68 with 95% confidence limits 0.54 to 0.81.

However, as with the islet index, the OCR alone proved to be of marginal value (P=0.10) in predicting post transplant function (FIG. 4).

### Example 5

### OCR Inhibition with Sodium Azide

Measuring a cells ability to consume oxygen (OCR) is an indicative means of assessing the metabolic potency of the mitochondria and overall function of the cell. Therefore, to correlate cellular oxygen consumption rates with mitochondrial potency, insulin-producing porcine islets were incubated with increasing concentrations of sodium azide (NaAz), a well known inhibitor of mitochondrial respiration, prior to OCR measurements. Wilson, D et al., “Azide Inhibition of Mitochondrial Electron Transport I: The Aerobic Steady State of Succinate Oxidation,” Biochim Biophys Acta 131, 421-430 (1967).

Reductions in OCR were assessed by comparing the effect of the NaAz on the OCR measurements to the OCR capability of non-treated cells. BTC6 cells and Hela cells were incubated on 150 mm culture dishes for 2 days in a 37° C. incubator with a 5% CO₂ environment. The cells were then removed using 5 mM EDTA/PBS and washed once with PBS. The resulting cell pellet was resuspended in 5 mL of DMEM (Sigma) supplemented with 5% FCS (Sigma) and Penicillin/Streptomycin (Invitrogen). Cells were then incubated with various concentrations (0.01, 0.1, 1.0 and 10 mg/ml) of sodium azide (Sigma) for 10 minutes prior to measuring their oxygen consumption rates.

To measure the effect of NaAz on the mitochondrial potency of islets, adult porcine islets were cultured overnight in modified RMPI (10%FBS, 5 mM Nicotinamide, 2 mM Glutamax, 1% P/S) at 37° C. with a 5% CO2 environment. On the day of the experiment, the islets were counted and washed using non-supplemented RMPI. The islets were then incubated with various concentrations (0.0, 0.1, 1.0 and 10 mg/ml) of sodium azide (Sigma) for 10 minutes to inhibit aerobic respiration prior to measuring their oxygen consumption rates. As a negative control, RMPI alone was tested for background oxygen consumption ability. The islets's subsequent OCR measurements were standardized to a percent difference from non-NaAz treated cells. Each OCR measurement per sodium azide treatment was performed in triplicate on 1000 IEQ per assay. The experiment was repeated 3 times.

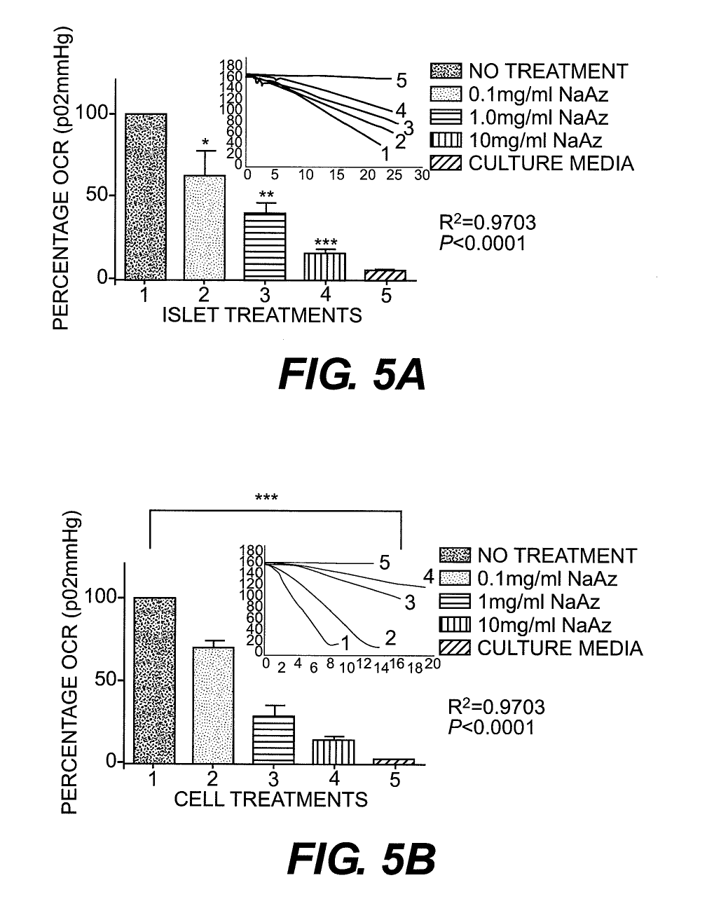

When islets were incubated with 0.1 mg/ml NaAz, a 37.6% reduction in OCR was achieved. Cells treated with NaAz at concentrations of 1 mg/ml, 10 mg/ml, and culture media alone resulted in OCR reductions of 59.7%, 84.2%, and 94.7%, respectively (FIG. 5A). Overall, NaAz significantly reduced the islets's ability to consume oxygen in a dose dependent manner (*p<0.05, **p<0.01, ***p<0.001).

To further support these observations, the above experiment was reproduced in βTC6 and Hela. When β-TC6 cells were incubated with 0.1 mg/ml NaAz, a 29.8±4.5% (n=4) reduction in OCR was achieved. Cells treated with NaAz at concentrations of 1 mg/ml, 10 mg/ml and culture media with 10 mg/ml resulted in OCR reductions of 71.2±6.6% (n=4), 85.9±2.3% (n=4) and 98.6±0.37% (n=4), respectfully (FIG. 5B). Overall, NaAz significantly reduced the OCR in β-TC6 cells (p<0.0001), and an overall dose dependent correlation of R²=0.9703 (FIG. 5B). Photomicrographs of Beta-TC6 cells with Mitotracker red and the EGFP-actin fluorescent were taken to further illustrate the mitochondrial damage induced by NaAz (data not shown).

The above results support the conclusion that oxygen consumption is a robust and specific indicator of metabolic activity as measured through mitochondrial potency and, for islets, and thus the ability to produce insulin and/or reverse diabetes post transplantation.

### Example 6

### Oxygen Consumption Rates Standardized to Islet Index

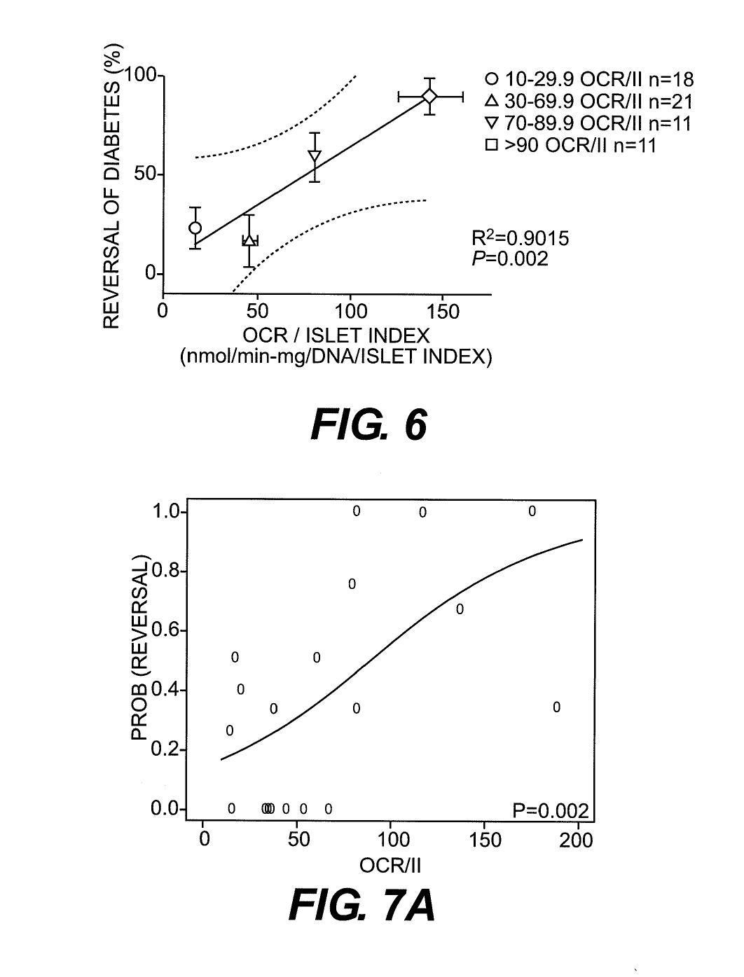

To account for the influence of islet size on their ability to consume oxygen, the OCRs of the islets were standardized to their islet index by generating an OCR/II ratio (standardized OCR). Islets were sub-divided into four groups based on this ratio: OCR/II from 10-29 (n=18), OCR/II from 30-69 (n=21), OCR/II from 70-89 (n=11) and OCR/II>90 (n=10). When islets had a low standardized OCR between 10-29, (16.726±1.075) and between 30-69, (46.021±3.763) reversal rates were 23.0% and 16.6%, respectively. Islet isolations that had higher standardized OCR ranging between 70-89, (80.49±1.03) nude mouse diabetes reversal rates increased to 58.3%. Finally, when standardized OCR values exceeded 90 (142.72±17.07), diabetes was reversed in 90% of the animals transplanted. Pre-transplant standardized OCR correlated strongly with diabetes reversal rates, (R²=0.9015). These data also indicate that the standardized OCR ratio is a highly statistically significant (P=0.002) pre-transplant indicator of post transplant function (FIG. 6).

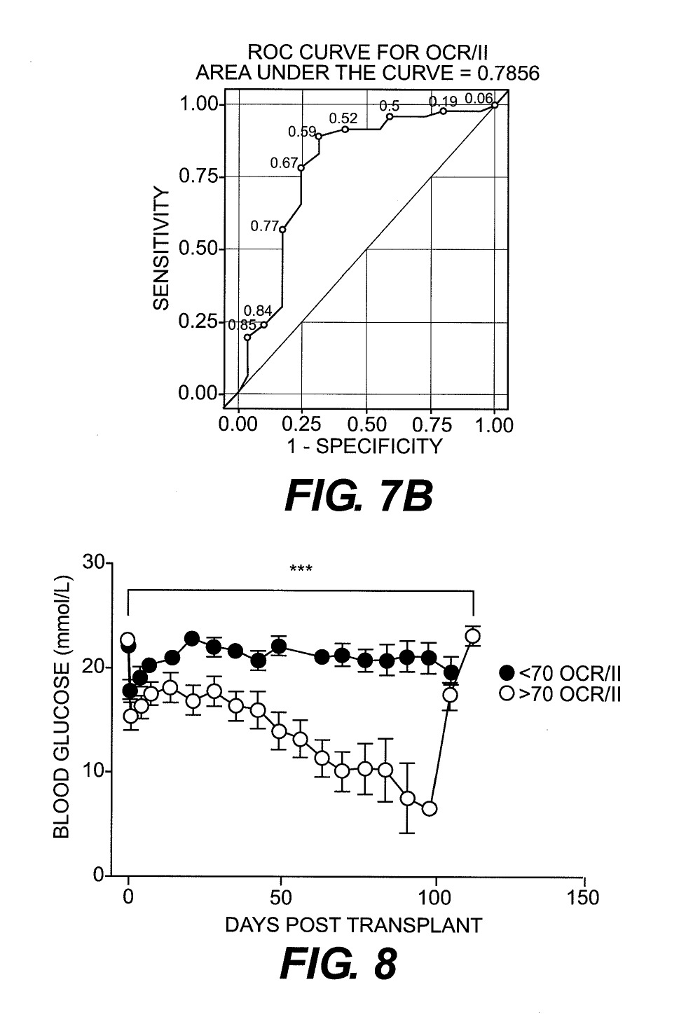

Logistic regression analysis of pre-transplant OCR/Islet indexes from 20 islet isolations showed a strong correlation (P=0.002) with diabetes reversal rates in 75 nude mouse transplants (FIG. 7A). Comparison of the regression lines between FIGS. 1 and 4 provides a visual guide to the beneficial effect of combining the OCR/DNA test with the islet index. This analysis also provides a useful mathematical relationship between the probability that islets will function in vivo and any standardized OCR index. The equation that describes this relationship is given by Pr (reversal)=1 over (1+exp (2.4030−0.033*OCR Index). ROC analysis (FIG. 7B) confirmed the value of this test. Results from 75 transplants produce an AUC of 0.79 with 95% confidence limits of 0.67 to 0.90 (P=0.0002).

### Example 7

### Summary of Results for Certain Assays Compared to Assessment Using Oxygen Consumption Rates Standardized to Islet Index (OCR/II)

The individual predictive ability of staining viability, glucose stimulation index, islet size, oxygen consumption rate, oxygen consumption rates/islet index was assessed using a logistic regression model with probability of diabetic reversal as response. The extent that a predictor distinguishes reversal and non-reversal was investigated using the receiver operating characteristic curve (ROC) methodology (Altman et al., “Statistics Notes: Diagnostic Tests 3: Receiver Operating Characteristic Plots” BMJ 309: 188 (1994)). An ROC plot is obtained by calculating the sensitivity and specificity of every observed data value and plotting sensitivity against 1 minus the specificity. The effectiveness of a predictor is then quantified by the area under the ROC curve, with the area of 0.5 indicating a useless predictor and 1.0 indicating a perfect predictor. P-value of 0.05 was used to declare the statistical significance of an index in predicting probability of reversal. All analyses were performed using SAS 9.2 (SAS institute, Cary, N.C.), other statistical software can be applied. As a result, the OCR/II value proved to be the greatest and most sensitive predictor of in vivo function (Table 1).

TABLE 1
Statistical analysis correlating in vitro functional
tests to reversal rates of diabetes

| | | Standard | 95% Confidence | | |
|---|---|---|---|---|---|
| Viability Model | Area | Error | Limits | P-Value | N |
| Viability Stain | 0.59 | 0.09 | 0.42-0.76 | 0.30 | 45 |
| Insulin Stim Index | 0.54 | 0.09 | 0.37-0.71 | 0.23 | 53 |
| Islet Index | 0.63 | 0.07 | 0.49-0.76 | 0.11 | 73 |
| OCR/DNA | 0.68 | 0.07 | 0.54-0.81 | 0.004 | 77 |
| OCR/II | 0.79 | 0.06 | 0.67-0.90 | 0.0002 | 75 |

Transplant recipients (no exclusions) were re-classified into two groups based upon their standardized OCR ranges, either <70 (n=50) or >70 (n=25). Islets with a standardized OCR value >70 were significantly better at reducing blood glucose levels in diabetic nude mice compared to those with values <70 (p<0.0001), indicating that a standardized OCR of 70 could be a valuable pre-transplant functional threshold (FIG. 8).

Based on these results, a threshold value of 70 for this factor was derived, above which there is a high probability of diabetes reversal. This assumption is based on the outcome of a logistic regression, which indicated that the probability of reversal diabetes increases with an increase in standardized OCR (P=0.002). Thus there is a relationship between the probability that islets will function in vivo and any given standardized OCR index. The equation to describe this relationship is given by Pr(reversal)=1 over (1+exp (2.4030−0.033 Index). Deriving this standardized OCR requires approximately 1000 to 3000 IEQ, is inexpensive, and can be measured in less than 30 minutes.

### Example 8

### Oxygen Consumption Rates Standardized to Islet Index in a Porcine Model

To validate the observations made in nude mice xenografts (pig to mouse) that an increase in oxygen consumption/islet index (OCR/II) correlated to a greater probability of reversing diabetes, islet OCR/II values were measured prior to islet auto-transplantation in a porcine model. Juvenile porcine islets were isolated from female (12-16 week old) Yorkshire-Landrace pigs using a modified Ricordi technique (Ricordi, C. et al. “A Method for the Mass Isolation of Islets from the Adult Pig Pancreas,” Diabetes, 35(6):649-53 (1986)) yielding >90% purity from exocrine tissue. Immature porcine islets were cultured for 5 days to allow animal to recover fully from pancreatectomy and ensure a stringent diabetic state was established, in modified RMPI (10%FBS, 5 mM Nicotinamide, 2 mM Glutamax, 1% P/S) at 37° C.

Approximately, 8,000 immature islet equivalents were auto-transplanted following pancreatectomy into pre-vascularized (2-8 weeks) subcutaneous devices. Before implantation, islet indices and OCRs were measured as described in Examples 3 and 4 above. Animals were monitored for graft function through weekly fasting and non-fasting blood glucose measurements and glucose and c-peptide responses to monthly intravenous glucose tolerance tests. In this model, islet graft function was defined as a reduction in daily blood glucose measurements, improved glucose, and C-peptide responses to an intravenous glucose tolerance test compared to pre-transplant and post device removal measurements.

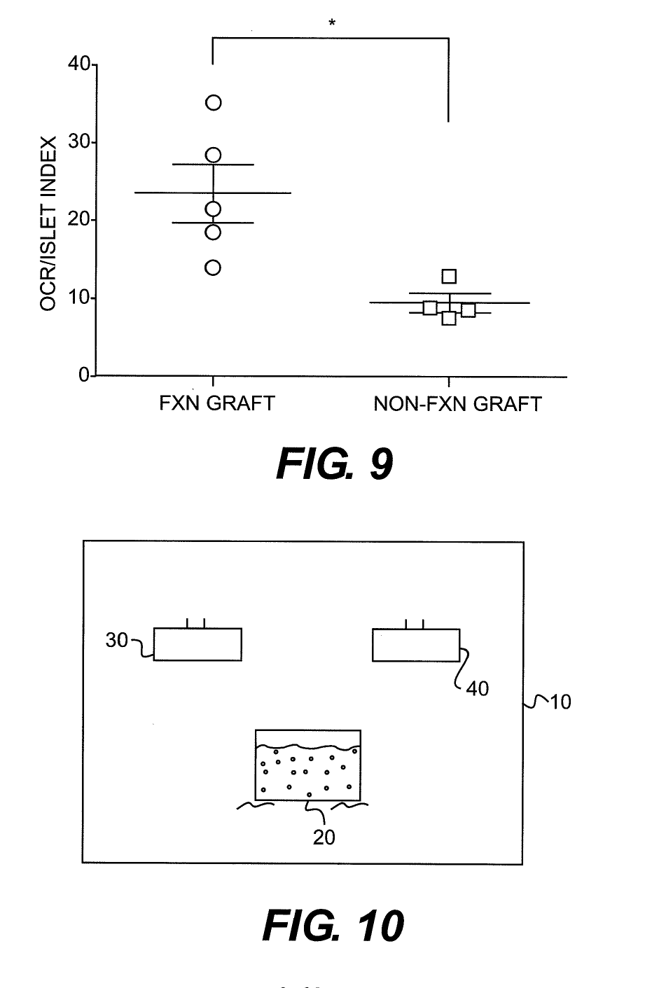

As shown in FIG. 9, an increase in OCR/II correlates with a greater probability of porcine islet engraftment and function, as measured though C-peptide secretion and response to an intravenous glucose tolerance test (post auto-transplantation). Further, as shown, these transplants, which included immature islets, not adult islets, produced functioning grafts when the OCR/II ratios were less than 50 nmol/min-mgDNA.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims

1. A method of assessing islet cell function, comprising:
determining an oxygen consumption rate (OCR) of a group of islet cells;
determining an islet index (II) of the islet cells; and
determining an OCR/II ratio, wherein a high OCR/II ratio correlates with improved islet function in vivo.

2. The method of claim 1, further comprising determining whether or not the islets are suitable for implantation in a host based on the OCR/II ratio.

3. The method of claim 1, wherein the host is a human.

4. The method of claim 1, wherein the host is a non-human animal.

5. The method of claim 1, wherein islets having an OCR/II ratio within a range of about 50 nmol/min-mg DNA to about 250 nmol/min-mgDNA are identified as being suitable for transplantation.

6. The method of claim 1, wherein islets having an OCR/II ratio of about 50, 60, 70, 80, 90 nmol/min-mg DNA or more are identified as being suitable for transplantation.

7-10. (canceled)

11. The method of claim 1, wherein the OCR/II ratio is determined by dividing the number of islet equivalents (IEQ) in a sample by the actual number of islets in the sample.

12. The method of claim 1, wherein the method is performed prior to transplanting islets into a recipient.

13. (canceled)

14. The method of claim 1, wherein islet function is measured as the ability of the islets to reverse diabetes in a diabetic recipient of an islet transplant and/or as increased probability of the ability of the islet to reverse diabetes in a diabetic recipient of an islet transplant.

15. The method of claim 14, wherein reversal of diabetes comprises production of at least one of a non-fasting blood glucose below 11.1 mmol/L or a fasting blood glucose 7.8 mmol/L post transplant.

16. The method of claim 1, wherein islet function is measured as the ability of an islet transplant to improve blood glucose levels in a diabetic recipient of an islet transplant.

17. The method of claim 16, wherein improved blood glucose comprises at least one of: a reduction in Hemoglobin A1C (HbA1C) compared to pre-transplant values; an increase in serum C-peptide concentrations compared to pre-transplant values; an improved response to a glucose tolerance test as measured by area under curve for glucose; insulin; and c-peptide; and glucose disappearance rates.

18-19. (canceled)

20. The method of claim 14, wherein the probability that an islet transplant will reverse diabetes post-transplant is given by the equation: Pr(reversal)=1 over (1+exp(2.4030−0.033 Index).

21. The method of claim 1, wherein determining an OCR of a group of islet cells comprises placing the islets in an area having a known oxygen concentration and measuring the change in oxygen concentration over time.

22. The method of claim 1, wherein the islets comprise at least one of human islets, non-human animal islets, stem cells, genetically modified cells, or any cell which releases insulin.

23. The method of claim 1, wherein the islets comprise porcine islets.

24. The method of claim 1, wherein the islets are adult islets.

25-35. (canceled)

36. The method of claim 1, wherein the portion of the group of islet, cells comprises 2,000 IEQ or fewer.

37-39. (canceled)

40. A device for assessing islet cell function, comprising:
a first cell analysis unit configured to determine an oxygen consumption rate (OCR) of a group of islet cells;
a second cell analysis unit configured to determine an islet index (II) of the islet cells; and
a computation unit configured to determine an OCR/II ratio, wherein a high OCR/II ratio correlates with improved islet function in vivo.

41. The device of claim 40, wherein the first cell analysis unit comprises an oxygen sensor.
